(19) United States
(12) Patent Application Publication
(10) Pub. No.: US 2026/0048268 A1
(43) Pub. Date: Feb. 19, 2026

(54) WAVEFORM EDITOR FOR NEUROMODULATION SYSTEMS

(71) Applicant: AJ Robotics LLC, Agua Dulce, CA (US)

(72) Inventors: Dongchul Lee, Agua Dulce, CA (US); Emarit Ranu, Fort Collins, CO (US)

(73) Assignee: AJ Robotics LLC, Agua Dulce, CA (US)

(21) Appl. No.: 19/279,783

(22) Filed: Jul. 24, 2025

Related U.S. Application Data
(60) Provisional application No. 63/683,330, filed on Aug. 15, 2024.

(51) Int. Cl. A61N 1/372; A61N 1/36

(52) U.S. Cl. CPC A61N 1/37247; A61N 1/3615; A61N 1/36171; A61N 1/36175

(57) ABSTRACT

A computing system for programming stimulation parameters of a neuromodulation system is disclosed. The computing system includes a graphical user interface configured to display a multidimensional parameter space, wherein each axis of the parameter space corresponds to a respective stimulation parameter associated with stimulation pulses deliverable by the neuromodulation system, and render a target within the multidimensional parameter space, the target representing a set of stimulation parameters corresponding to coordinates along two or more axes of the multidimensional parameter space. The system further includes one or more user-operable controls configured to enable movement of the target within the multidimensional parameter space. Movement of the target within the multidimensional parameter space causes simultaneous adjustment of the stimulation parameters associated with the neuromodulation system along the two or more axes based on a position of the target in the multidimensional parameter space.

### CROSS-REFERENCE TO RELATED APPLICATIONS

This application claims the benefit of U.S. Provisional Application No. 63/683,330, filed Aug. 15, 2024, which is incorporated by reference herein.

### FIELD

The present disclosure concerns systems and methods for programming stimulation parameters in neuromodulation systems.

### BACKGROUND

Neuromodulation systems deliver electrical modulation of electrically-active tissues to manage conditions such as chronic pain and neurological disorders. Programming these systems requires adjustment of multiple stimulation parameters, such as pulse width, amplitude, frequency, etc. Conventional programming interfaces often rely on manual, sequential tuning of individual parameters, making the process inefficient and difficult to optimize. Clinicians and patients may also lack intuitive tools for exploring how combinations of parameters affect therapeutic outcomes. Thus, there is room for improvement in neuromodulation programming systems to provide more efficient, user-friendly, and responsive tools for parameter selection.

### SUMMARY

Described herein are systems and methods for programming stimulation parameters in neuromodulation systems using a multidimensional graphical user interface, which overcome one or more of the deficiencies of conventional parameter programming technologies. While examples throughout this disclosure may refer to neuromodulation or stimulation of neural tissue, the disclosed technologies are more broadly applicable to electrical modulation of electrically-active tissues, including but not limited to neural, muscular, and other excitable tissues. Accordingly, references to neuromodulation should be understood as encompassing electrically-active tissue modulation more generally.

Certain aspects of the disclosure concern a computing system for programming stimulation parameters of a neuromodulation system. The computing system includes a graphical user interface configured to display a multidimensional parameter space, wherein each axis of the multidimensional parameter space corresponds to a respective stimulation parameter associated with stimulation pulses deliverable by the neuromodulation system, and render a target within the multidimensional parameter space. The target represents a set of stimulation parameters corresponding to coordinates along two or more axes of the multidimensional parameter space. The computing system further includes one or more user-operable controls configured to enable movement of the target within the multidimensional parameter space. Movement of the target within the multidimensional parameter space causes simultaneous adjustment of the stimulation parameters associated with the neuromodulation system along the two or more axes based on a position of the target in the multidimensional parameter space.

Certain aspects of the disclosure concern a computer-implemented method for programming stimulation parameters of a neuromodulation system. The method includes displaying, via a graphical user interface, a multidimensional parameter space. Each axis of the multidimensional parameter space corresponds to a respective stimulation parameter associated with stimulation pulses deliverable by the neuromodulation system. The method also includes rendering, within the multidimensional parameter space, a target representing a set of stimulation parameters corresponding to coordinates along two or more axes of the multidimensional parameter space; receiving, via one or more user-operable controls, input to move the target within the multidimensional parameter space; and in response to movement of the target, simultaneously adjusting the stimulation parameters associated with the neuromodulation system along the two or more axes based on a position of the target in the multidimensional parameter space.

Certain aspects of the disclosure concern one or more non-transitory computer-readable media having encoded thereon computer-executable instructions causing one or more processors to perform a method for programming stimulation parameters of a neuromodulation system. The method includes displaying, via a graphical user interface, a multidimensional parameter space. Each axis of the multidimensional parameter space corresponds to a respective stimulation parameter associated with stimulation pulses deliverable by the neuromodulation system. The method further includes rendering, within the multidimensional parameter space, a target representing a set of stimulation parameters corresponding to coordinates along two or more axes of the multidimensional parameter space; receiving, via one or more user-operable controls, input to move the target within the multidimensional parameter space; and in response to movement of the target, simultaneously adjusting the stimulation parameters associated with the neuromodulation system along the two or more axes based on a position of the target in the multidimensional parameter space.

Although primarily described in the context of programming stimulation parameters for neuromodulation systems, it should be understood that the systems and methods disclosed herein may also be applied to other types of stimulation devices that utilize configurable electrical pulse parameters. For example, the disclosed graphical user interface and control mechanisms may be used to program parameters for functional electrical stimulation devices used in rehabilitation therapy, transcutaneous electrical nerve stimulation devices used for pain management, implantable cardiac pacemakers and/or cardioverter-defibrillators, etc. More generally, the disclosed technologies may be adapted for any device that benefits from user-guided, multidimensional exploration of stimulation settings.

The foregoing and other features and advantages of the disclosed technologies will become more apparent from the following detailed description, which proceeds with reference to the accompanying figures.

### BRIEF DESCRIPTION OF THE DRAWINGS

FIG. 1 is a schematic view of an example electrical stimulation system including a paddle lead coupled to a control module.

FIG. 2 is a schematic view of an example electrical stimulation system including a percutaneous lead coupled to a control module.

FIG. 3A is a schematic view of an example control module configured to couple to an elongated device.

FIG. 3B is a schematic view of an example lead extension configured to couple an elongated device to a control module.

FIG. 4 is a schematic overview of example components of a stimulation system, including an electronic subassembly within a control module.

FIG. 5 is an overall block diagram depicting components of a programming device for a neuromodulation system, according to one example.

FIG. 6 is a flowchart illustrating an example overall method for programming stimulation parameters of a neuromodulation system.

FIG. 7 depicts an example graphical user interface for simultaneous adjustment of two stimulation parameters in a two-dimensional parameter space.

FIG. 8 depicts an example graphical user interface depicting multiple zones within a two-dimensional parameter space.

FIG. 9 depicts an example graphical user interface depicting a plurality of targets within a two-dimensional parameter space and example user-operable controls.

FIG. 10 depicts an example graphical user interface depicting a plurality of targets within a three-dimensional parameter space and example user-operable controls.

FIG. 11 is a block diagram of an example computing system in which described technologies can be implemented.

### DETAILED DESCRIPTION

### Overview of Neuromodulation Systems

Neuromodulation systems are medical technologies that deliver electrical modulation to electrically-active tissues of the body, such as the neural tissue and/or muscle tissue, to alleviate symptoms of various conditions, such as chronic pain, movement disorders, neurological dysfunctions, etc. These systems typically include a control module (e.g., a pulse generator), one or more leads with electrodes for delivering stimulation, and programming software that allows clinicians and/or patients to configure the stimulation parameters. The effectiveness of neuromodulation therapy depends in part on the proper selection and adjustment of stimulation parameters such as pulse width, amplitude, frequency, waveform shape, phase timing and characteristics, etc.

Programming a neuromodulation system involves navigating a multidimensional space of parameter settings to identify combinations that produce the desired physiological and therapeutic effects. Conventionally, programming interfaces allow users to modify parameters individually through sequential inputs such as drop-down menus, sliders, increment/decrement buttons, or the like. This approach can be time-consuming, unintuitive, and inefficient, especially when multiple interdependent parameters must be tuned in coordination. Moreover, it is difficult for clinicians and patients to visualize the parameter space and/or to understand how changes in one parameter affect others and the overall therapeutic outcome.

Another challenge in conventional systems is the lack of guidance during programming. While some systems allow for basic trial-and-error tuning, they do not typically offer real-time feedback tied to physiological models, nor do they provide visual indicators that reflect likely outcomes, such as optimal stimulation regions and/or thresholds to avoid. As a result, programming often requires significant expertise, subjective patient input, and multiple iterative sessions, leading to delays in achieving effective therapy and suboptimal use of device capabilities.

The technology described herein provides a more intuitive and efficient approach to programming neuromodulation systems using a multidimensional graphical user interface (GUI) that enables real-time exploration and adjustment of multiple parameters simultaneously. As described more fully below, this solution offers enhanced visual feedback, streamlined interaction, and intelligent guidance for selecting stimulation settings that improve ease of use, personalization, and therapeutic precision.

### Example Neuromodulation Systems

The present disclosure relates to electrical stimulation systems, and more particularly to neuromodulation systems that deliver therapeutic electrical stimulation (e.g., pulses) to neural, muscular, and/or other excitable tissue in the body. Although the examples provided herein primarily depict implantable neuromodulation systems, the disclosed technologies are also applicable to external or wearable devices. The stimulation pulses delivered by such systems can be programmed using the waveform editing and parameter programming technologies described herein. While stimulation pulses are used as illustrative examples throughout, it should be understood that the disclosed programming methodologies are also adaptable to non-pulsed stimulation modalities, which may include continuous waveforms or other non-discrete signal formats.

Suitable neuromodulation systems can include at least one lead having one or more electrodes disposed along its distal end and one or more terminals disposed along a proximal end. Leads may take the form of paddle leads, percutaneous leads, or cuff leads, and may be used with or without extensions, adaptors, or splitters. In some cases, a stimulation system can include an implantable microstimulator, where stimulation is delivered via electrodes integrated into the device housing.

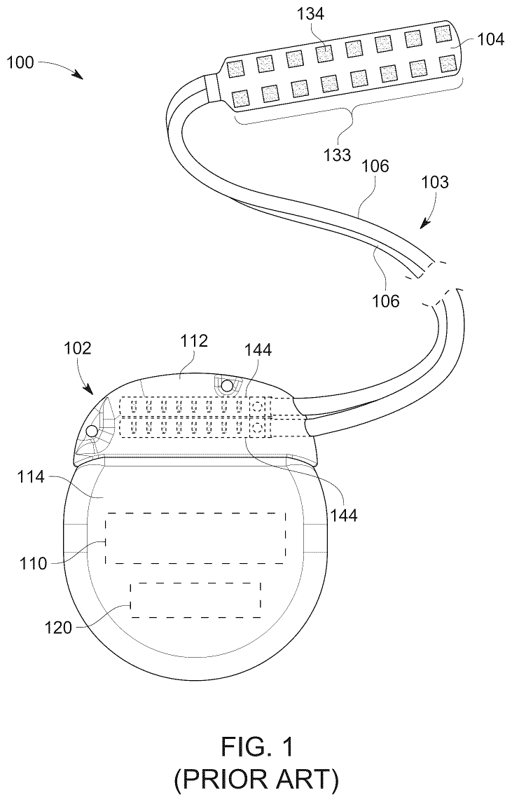

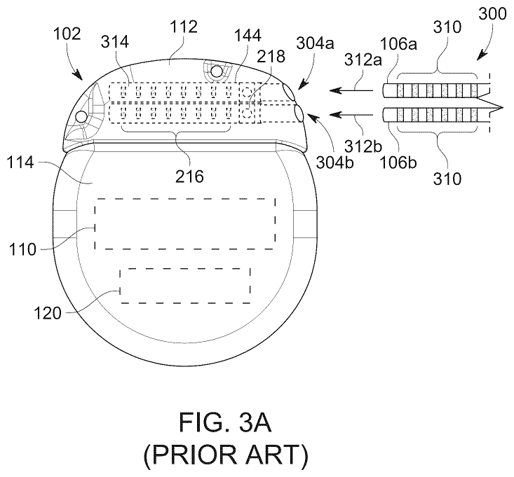

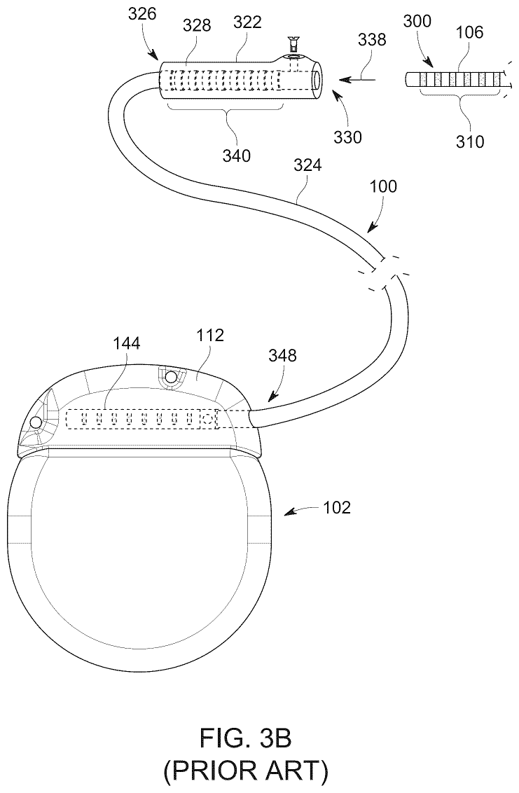

FIG. 1 schematically illustrates one example of an electrical stimulation system 100. The electrical stimulation system 100 includes a control module 102 (e.g., a stimulator or pulse generator) and a lead 103 that is coupleable to the control module 102. The lead 103 includes a paddle body 104 and one or more lead bodies 106. In the illustrated example, the lead 103 has two lead bodies 106, although any suitable number may be used, such as one, two, three, four, five, six, seven, eight, or more. An electrode array 133, including electrodes such as electrode 134, can be disposed on the paddle body 104. An array of terminals (e.g., 310 in FIGS. 3A and 3B) can be disposed along each of the one or more lead bodies 106.

It will be appreciated that the electrical stimulation system 100 may include additional, fewer, or alternative components, and may be implemented in a variety of configurations beyond those specifically illustrated. For example, instead of a paddle body, the electrodes may be arranged in an array at or near the distal end of a lead body to form a percutaneous lead. In some embodiments, a paddle lead may include only a single electrode, and similarly, a percutaneous lead may include just one electrode. Additionally, the control module itself may include one or more electrodes in certain configurations.

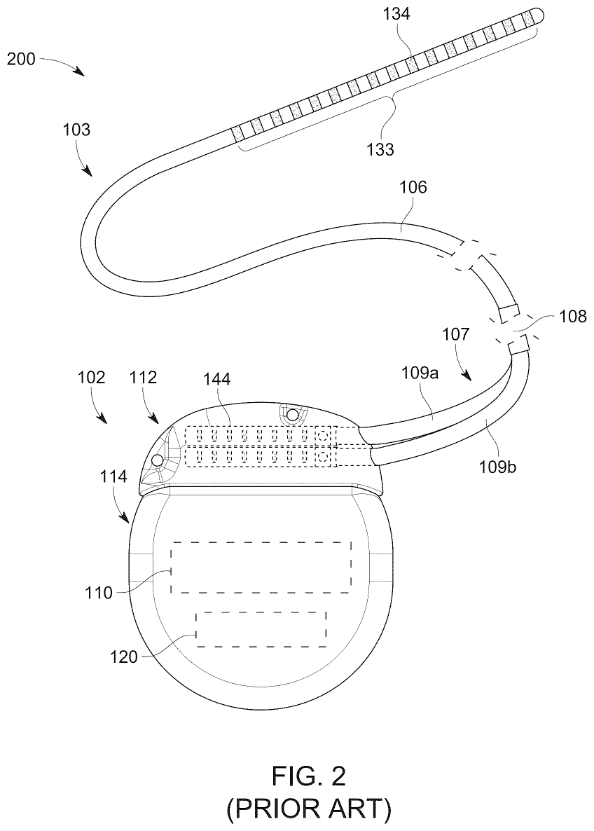

FIG. 2 schematically illustrates another embodiment of the electrical stimulation system 200, in which the lead 103 is configured as a percutaneous lead. In this example, electrodes 134 are shown disposed along one or more lead bodies 106. In at least some implementations, the lead 103 can be isodiametric along the longitudinal length of the lead body 106.

The lead 103 can be coupled to the control module 102 in any suitable manner. In the example shown in FIG. 1, the lead 103 is coupled directly to the control module 102. In other examples, the lead 103 may be connected to the control module 102 via one or more intermediate devices, such as a lead extension 324 illustrated in FIG. 3B. One or more lead extensions may be used to increase the distance between the lead 103 and the control module 102. Additionally, other intermediate components, such as splitters, adaptors, or combinations thereof, may be used in place of, or in addition to, lead extensions. Where multiple elongated devices are positioned between the lead 103 and the control module 102, the intermediate devices may be arranged in any configuration suitable for the specific clinical or system requirements.

In FIG. 2, the electrical stimulation system 200 includes a splitter 107 configured to facilitate coupling of the lead 103 to the control module 102. The splitter 107 includes a splitter connector 108 that connects to the proximal end of the lead 103, and one or more splitter tails 109a and 109b that are configured to couple to the control module 102, or alternatively to another splitter, lead extension, adaptor, or similar component.

With reference to FIGS. 1 and 2, the control module 102 typically includes a connector housing 112 and a sealed electronics housing 114. The electronics housing 114 contains an electronic subassembly 110 and, in some embodiments, an optional power source 120. A control module connector 144 can be disposed within the connector housing 112 and be configured to establish an electrical connection between the lead 103 and the electronic subassembly 110 of the control module 102.

The electrical stimulation system, or components thereof (e.g., the paddle body 104, one or more lead bodies 106, and the control module 102) can be implanted within a patient's body. The system may be used in a variety of therapeutic applications, including but not limited to deep brain stimulation, neural stimulation, spinal cord stimulation, muscle stimulation, and similar neuromodulation therapies.

The electrodes 134 may be formed from any conductive, biocompatible material. Suitable materials include metals, metal alloys, conductive polymers, conductive carbon, and combinations thereof. In some examples, one or more electrodes 134 can be made from materials such as platinum, platinum-iridium, palladium, palladium-rhodium, or titanium.

Any suitable number of electrodes 134 may be disposed on the lead, including, for example, one, two, three, four, five, six, seven, eight, nine, ten, eleven, twelve, fourteen, sixteen, twenty-four, thirty-two, or more. For paddle leads, the electrodes 134 can be arranged on the paddle body 104 in a variety of configurations. For example, in FIG. 1, the electrodes are arranged in two columns, with each column containing eight electrodes.

The electrodes on the paddle body 104 (or on one or more lead bodies 106) can be embedded in or separated by a non-conductive, biocompatible material such as silicone, polyurethane, polyetheretherketone (PEEK), epoxy, or combinations thereof. The lead bodies 106 and, where applicable, the paddle body 104 may be formed into the desired shape using manufacturing processes such as molding (including injection molding), casting, or similar techniques. The non-conductive material can extend along each lead body 106 from its distal end to its proximal end.

For paddle leads, the non-conductive material can extend from the paddle body 104 to the proximal end of each lead body 106. The biocompatible, non-conductive material used in the paddle body 104 and the lead bodies 106 may be the same or different. Additionally, the paddle body 104 and the lead bodies 106 may be formed as a single, unitary structure or as separate components that are permanently or detachably coupled together.

Terminals (e.g., 310 in FIGS. 3A and 3B) can be disposed along the proximal end of one or more lead bodies 106 of the electrical stimulation system 100 or 200, as well as on any intermediate components such as splitters, lead extensions, or adaptors. These terminals can provide electrical connections to corresponding connector contacts (e.g., 314 in FIG. 3A), which are housed within connectors (e.g., 144 in FIGS. 1-3B; 322 in FIG. 3B) located on the control module 102 or on associated intermediate devices. Electrically conductive wires, cables, or similar structures can extend from the terminals to the electrodes 134. In some examples, one or more electrodes 134 can be electrically coupled to each terminal. In some examples, each terminal is only connected to a single electrode 134.

The electrically conductive wires (or “conductors”) may be embedded within the non-conductive material of the lead body 106 or housed in one or more lumens extending along the length of the lead body. In some examples, each conductor is routed through a dedicated lumen, while in others, multiple conductors can share a common lumen. One or more lumens may open near the proximal end of the lead body 106 to allow insertion of a stylet, aiding in placement of the lead within the patient's body. Additionally, one or more lumens may open near the distal end to permit infusion of drugs or medication at the implantation site. In certain examples, the lumens can be flushed continuously or periodically with saline, epidural fluid, or similar substances. Some lumens may also be configured to be permanently or removably sealed (e.g., at the distal end).

FIG. 3A is a schematic side view of one embodiment of a proximal end of one or more elongated devices 300, configured to couple with the control module connector 144. The elongated devices 300 may include, for example, one or more lead bodies 106 shown in FIG. 1, as well as intermediate components such as a splitter, the lead extension 324 shown in FIG. 3B, an adaptor, or combinations thereof.

In some examples, the control module connector 144 defines at least one port for receiving a proximal end of the elongated device 300, as indicated by directional arrows 312a and 312b. In FIG. 3A, the connector housing 112 is shown with two ports, 304a and 304b. However, the connector housing 112 may define any suitable number of ports, including one, two, three, four, five, six, seven, eight, or more.

Each port 304a and 304b of the control module connector 144 can include a plurality of connector contacts, such as connector contact 314. When the elongated device 300 is inserted into these ports, the connector contacts 314 can align with a corresponding plurality of terminals 310 disposed along the proximal ends of the elongated device(s) 300. This alignment can establish an electrical connection between the control module 102 and the electrodes (e.g., electrodes 134 in FIG. 1) located on the paddle body 104 of lead 103.

FIG. 3B is a schematic side view of another embodiment of the electrical stimulation system 100 or 200. In this example, the system includes a lead extension 324 configured to couple one or more elongated devices 300, such as a lead body 106 (FIGS. 1 and 2), the splitter 107 (FIG. 2), an adaptor, another lead extension, or combinations thereof, to the control module 102. In FIG. 3B, the lead extension 324 is shown connected to a single port 304 of the control module connector 144 and arranged to couple with a single elongated device 300. In other examples, the lead extension 324 may be configured to connect to multiple ports 304 on the control module connector 144, to receive multiple elongated devices 300, or both.

In some examples, a lead extension connector 322 can be positioned on the lead extension 324. In FIG. 3B, the connector 322 is shown at the distal end 326 of the lead extension. The connector 322 includes a housing 328 that defines at least one port 330 for receiving terminals 310 of the elongated device 300, as indicated by directional arrow 338. Within the housing 328, a plurality of connector contacts, such as contacts 340, are provided. When the elongated device 300 is inserted into port 330, the connector contacts 340 can align with the terminals 310 to electrically couple the lead extension 324 to the electrodes (e.g., electrodes 134 of FIGS. 1 and 2) disposed along lead 103.

In some examples, the proximal end of the lead extension 324 can be configured similarly to the proximal end of the lead 103 (or another elongated device 300). The lead extension 324 may include a plurality of electrically conductive wires that extend from the connector contacts 340 to a proximal end 348, opposite the distal end 326. These conductive wires may be electrically coupled to a plurality of terminals disposed along the proximal end 348. In certain implementations, the proximal end 348 can be configured for insertion into a connector of another lead extension or intermediate device. In other examples, such as the example shown in FIG. 3B, the proximal end 348 can be configured for direct insertion into the control module connector 144.

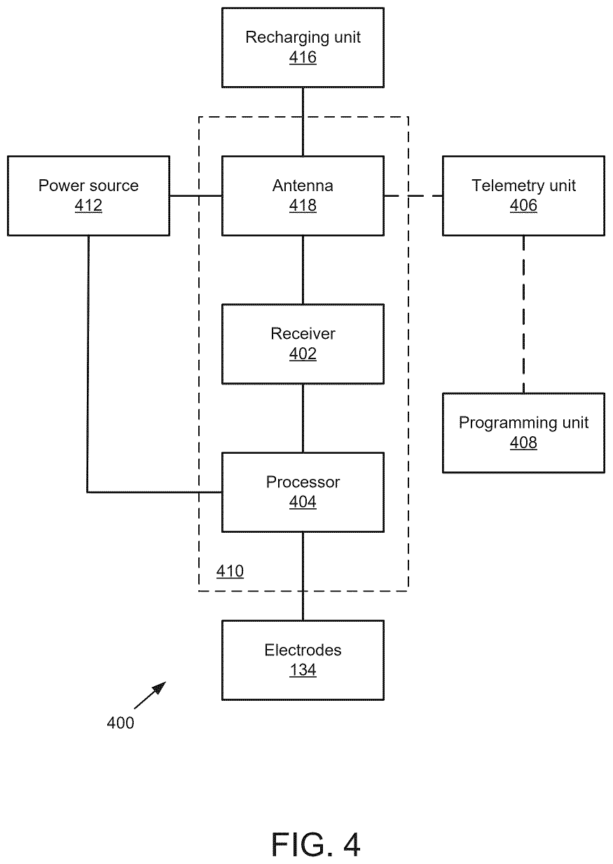

FIG. 4 is a schematic overview of components of an example electrical stimulation system 400, including an electronic subassembly 410 housed within a control module. It should be understood that the electrical stimulation system 400 may include additional, fewer, or alternative components, and can be implemented in a variety of configurations beyond those specifically illustrated.

Various components of the electrical stimulation system, such as a power source 412, an antenna 418, a receiver 402, and a processor 404, can be mounted on one or more circuit boards or similar carriers within a sealed housing of an implantable pulse generator or control module, or within the housing of a microstimulator. The power source 412 may be any suitable type, including primary or rechargeable batteries. Other possible power sources include supercapacitors, nuclear or atomic batteries, mechanical resonators, infrared collectors, thermally powered generators, flexural energy harvesters, bioenergy sources, fuel cells, bioelectric cells, and osmotic pressure pumps.

Alternatively, power may be supplied by an external source, e.g., via inductive coupling through the optional antenna 418 or a secondary antenna. The external power source may reside in a device worn on the user's skin or located nearby on a permanent or periodic basis.

If the power source 412 is a rechargeable battery, it can be recharged using the optional antenna 418. In such cases, power can be inductively transferred from an external recharging unit 416 to the battery via the antenna. This setup enables convenient wireless recharging without requiring direct physical connections.

In some embodiments, electrical current can be delivered through electrodes 134 located on a paddle, lead body, or microstimulator housing to stimulate nearby nerve fibers, muscle fibers, or other excitable tissues. The processor 404 typically manages the timing and electrical characteristics of the stimulation. For example, the processor 404 may control parameters such as pulse timing, frequency, amplitude, duration, waveform shape, etc. It can also select which electrodes are active during stimulation and determine their polarity, e.g., assigning specific electrodes as cathodes or anodes. In certain implementations, the processor 404 can further identify electrode configurations that yield optimal stimulation of the target tissue.

The processor may range from a simple electronic device that generates pulses at regular intervals to a more sophisticated unit capable of receiving and interpreting instructions from a programming unit 408 (also referred to as a “programmer”). For example, such instructions may enable modification of pulse characteristics. In the illustrated embodiment, the processor 404 is connected to a receiver 402, which is further coupled to the optional antenna 418. This configuration allows the processor 404 to receive external commands for controlling parameters such as pulse attributes and electrode selection, as desired. In some examples, the programming unit 408 can be deemed as part of the electrical stimulation system 400. In some examples, the programming unit 408 can be deemed as external to the electrical stimulation system 400. In some examples, the programming unit 408 can be configured to implement the waveform editing and multi-dimensional parameter programming features, as described more fully below.

In some examples, the antenna 418 can be configured to receive signals (e.g., RF signals) from an external telemetry unit 406, which can be programmed by the programming unit 408. The programming unit 408 may be integrated into or separate from the telemetry unit 406. The telemetry unit 406 can take the form of a wearable device, such as one mounted on the user's skin, or a portable device resembling a pager, cellular phone, or remote control. Alternatively, the telemetry unit 406 may be a stationary device located at a home station or clinician's office. The programming unit 408 may be any suitable system capable of supplying data to the telemetry unit 406 for transmission to the electrical stimulation system 400. This communication may occur via a wired or wireless connection. One example of a suitable programming unit is a computer operated by a clinician or user to send programming commands to the telemetry unit 406.

The signals received by the processor 404 via the antenna 418 and receiver 402 can be used to control or adjust the operation of the electrical stimulation system. These signals may instruct the system to modify stimulation pulse characteristics, including pulse duration, frequency, waveform, or amplitude. In addition, the signals may command the system to initiate or cease stimulation, begin or stop battery charging, or perform other control functions. In alternative embodiments, the stimulation system may be configured without an antenna 418 or receiver 402, in which case the processor 404 operates according to pre-programmed parameters.

Optionally, the electrical stimulation system 400 may include a transmitter coupled to the processor 404 and antenna 418 to facilitate outgoing communication to the telemetry unit 406 or another receiving device. For instance, the system may transmit operational status updates, battery charge levels, or notifications of malfunction. The processor 404 may also transmit information about stimulation parameters, allowing the user or clinician to review or verify current settings.

Stimulation energy can be delivered from the control module 102 to the patient's tissue via the lead 103 and its electrodes 134, or alternatively, from a microstimulator through electrodes disposed on the housing of the microstimulator.

Stimulation energy can be delivered in the form of a series of electrical pulses, each characterized by specific properties. These pulses can be defined by stimulation parameters such as pulse amplitude and pulse frequency (or pulse period), as well as by the selection of one or more electrodes 134 for delivering the stimulation energy. In some embodiments, the pulse amplitude may range from 0 to 25 mA, or from 0 to 50 mA. The pulse frequency may range from 1 Hz to about 15,000 Hz, including narrower ranges such as 1 to 2,000 Hz or 1 to 1,200 Hz, depending on the application.

The stimulation configuration may include one or more electrodes designated as anodes and one or more electrodes designated as cathodes, in any suitable combination. For example, some embodiments can specify a single pulse amplitude and frequency, with specific electrodes identified as either anode or cathode to define the polarity of stimulation. In other embodiments, more complex pulsing schemes may be employed to deliver tailored stimulation patterns. These stimulation pulse schemes are typically configured to be charge-balanced (i.e., the net electrical charge delivered during the pulse is approximately zero) to ensure safe and effective delivery of stimulation.

### Example Computing System for Programming Stimulation Parameters

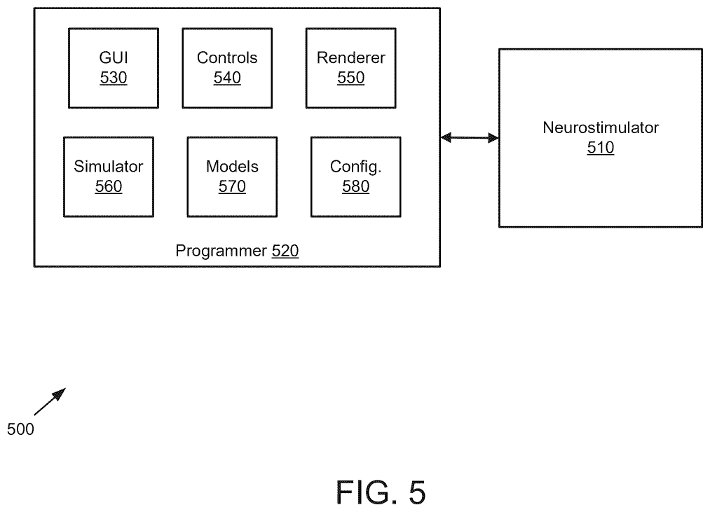

FIG. 5 illustrates an example neuromodulation system 500 that includes a neurostimulator 510 and a programmer 520. The programmer 520 may be an example embodiment of the programming unit 408 described above with respect to FIG. 4, and the neurostimulator 510 may correspond to the remaining components of the electrical stimulation system 400, excluding the programming unit. The programmer 520 can be configured to communicate with the neurostimulator 510 to program stimulation parameters (e.g., editing stimulation waveform), retrieve data, and optionally perform system diagnostics or closed-loop functions. Communication between the programmer 520 and neurostimulator 510 may be unidirectional or bidirectional and may be implemented via wired or wireless communication protocols.

The programmer 520 includes several functional modules to facilitate stimulation programming. In the depicted example, the programmer 520 includes a graphical user interface (GUI) 530, one or more user-operable controls 540, a renderer 550, a simulator 560, one or more physiological models 570, and a configuration storage module 580. As described more fully below, these modules work together to enable intuitive navigation of a multidimensional parameter space, real-time visualization of stimulation effects, and intelligent guidance based on physiological modeling. Although shown as separate functional blocks, one or more of these modules may be combined into a single component or distributed across multiple subsystems. Additionally, the programmer 520 may include other modules not shown (e.g., a telemetry interface for communicating with the neurostimulator 510, etc.).

The GUI 530 is configured to display a multidimensional parameter space in which each axis represents a stimulation parameter, such as total pulse width, relative phase duration, amplitude, frequency, pulse shape, interphase characteristic, or other programmable aspects of the stimulation waveform. The GUI 530 serves as the primary point of interaction between a user (e.g., a physician, a clinical technician, or the patient) and the programming system, providing both control functionality and visual feedback. The GUI 530 may present real-time graphical representations of a target's location within the parameter space, modeled zones of interest (if any), waveform previews, and active stimulation settings. As described herein, a “target” represents a specific combination of stimulation parameters, with its position in the multidimensional parameter space defining the values of those parameters along the respective axes. In some examples, the GUI 530 may also include interactive elements such as sliders, buttons, or icons for adjusting parameters or saving configurations. The GUI 530 can be rendered on a variety of display devices, including touchscreens, monitors, or mobile displays, and may support both clinician-facing and patient-facing views.

The user-operable controls 540 are configured to enable a user to interact with the GUI 530 and manipulate the stimulation parameters represented within the multidimensional parameter space. These controls may include input mechanisms such as a touchscreen interface, physical buttons, knobs, sliders, trackballs, joysticks, keyboards, and/or pointing devices like a mouse or stylus. In some embodiments, the controls 540 may be integrated directly into the GUI 530, for example, as on-screen touch controls on a tablet or mobile device. In other embodiments, the controls may be separate from the GUI 530, such as a dedicated input console or peripheral device. The user-operable controls 540 allow the user to move one or more targets within the parameter space to simultaneously adjust multiple stimulation parameters, select and edit previously saved parameter sets, toggle between display modes, or provide other user input. In some embodiments the controls 540 may include alternative input technologies that can “translate” users' intent to input. For instance, the controls 540 may include brain-computer interfaces (BCIs), which allow users to navigate the parameter space through neural commands, e.g., using electroencephalography (EEG)-based input. As another example, the controls 540 may incorporate three-dimensional limb or hand tracking systems, enabling users to manipulate targets within the parameter space using physical gestures.

The renderer 550 can be configured to generate visual representations of stimulation parameters (and in some implementations, associated effects) within the GUI 530. For example, the renderer 550 may synthesize and display one period of stimulation waveform (also referred to as a “wavelet”) based on selected parameter settings, visually indicating attributes such as pulse width, amplitude, relative phase durations, interphase delay, pulse shape, etc. These graphical outputs provide intuitive feedback that can help the user to understand the relationship between parameter adjustments and resulting stimulation characteristics. In some examples, the renderer 550 may also depict the position and movement of the target within the multidimensional parameter space, update dynamic zone overlays generated by the simulator 560, or visually highlight active or selected parameter sets. In some examples, the renderer 550 may incorporate color coding, animation, or other cues (e.g., brightness, contrast, hues, etc.) to enhance clarity and support real-time interactivity. The visual outputs produced by the renderer 550 enable users to evaluate and refine stimulation configurations before programming them into the neurostimulator 510.

The models 570 represent computational constructs that simulate the physiological response of biological tissues, such as nerve fibers, to electrical stimulation under varying conditions. These models 570 can be grounded in empirical data, mechanistic biophysics, or learned approximations from clinical datasets. In some examples, the models 570 may be used to estimate stimulation effects such as activation thresholds, recruitment selectivity, evoked compound action potentials (ECAPs), therapeutic efficacy, and potential side effects. For instance, the Hodgkin-Huxley (HH) model can be employed for simulating unmyelinated C-fibers, which are implicated in pain modulation, mechanoception and opioid-mediated responses. In another example, the McIntyre-Richardson-Grill (MRG) model can be used to simulate the electrical behavior of A-sized fibers, which typically have lower activation thresholds and are more susceptible to acute stimulation. Other models can also be incorporated.

The simulator 560 can be configured to execute the models 570 to evaluate the effects of candidate stimulation settings across a multidimensional parameter space. In some implementations, the simulator 560 uses numerical solvers to compute axonal excitation thresholds, assess fiber recruitment selectivity, and simulate response behaviors such as firing probability and pattern stability. Based on simulation outputs, the simulator 560 may generate predictive visualizations, such as zones or regions of interest that represent predefined stimulation effects. These may include a region where ECAPs are likely to be detected, a therapeutic efficacy zone (e.g., associated with selective large-fiber inhibition), or regions where specific neural activation patterns emerge, such as bursting or dual-spike behavior. In some examples, these zones of interest may be customized, modified, and/or deleted based on patient-reported outcomes or other conditions, enabling personalized optimization. In some examples, the programmer 520 may implement artificial intelligence (AI) agents or other intelligent algorithms that autonomously explore or adjust stimulation parameters based on patient-specific data.

As one specific (and non-exclusive) example, the simulator 560 can be used to generate a dual-firing zone (DFZ) by executing simulations using the MRG model within a neuronal simulation environment (e.g., NEURON). In this approach, the simulator 560 parametrically varies stimulation inputs across two dimensions: total pulse width (TPW) (e.g., ranging from 100 to 2000 μs) and relative stimulation phase (RSP) (e.g., ranging from 10% to 90%). Simulations are run across a range of axon diameters (e.g., 5.7 to 11.5 μm) using a bipolar electrode configuration (e.g., 5 mm inter-electrode spacing, positioned 10 mm from the nerve trunk). For each condition, the simulator 560 evaluates activation thresholds, response reliability, and efficiency. A dual-firing response can be identified when TPW exceeds approximately 1300 us and RSP falls between 40% and 70%, leading to the generation of two action potentials in response to a single stimulation pulse. This phenomenon occurs due to re-excitation triggered by the delayed cathodic recharge phase of the waveform. The resulting DFZ, mapped within the stimulation parameter space, can be displayed on the GUI 530 to inform the user of regions associated with this firing behavior.

In addition to, or in lieu of, zones associated with neural activation patterns, the simulator 560 may also be configured to define zones/regions of interest based on other physiological metrics, which can be calculated via modeling or derived from direct measurements. For example, the simulator 560 may identify zones/regions corresponding to stimulation-induced cardiac responses (e.g., bradycardia, tachycardia, change of blood pressure, etc.), muscle activity patterns (e.g., electromyography readings), and/or patient-reported side effects (e.g., nausea, vertigo, or disequilibrium). These zones can be rendered within the stimulation parameter space and used to guide the programming process, e.g., either by steering stimulation away from zones/regions associated with adverse responses or toward zones/regions correlated with desirable physiological outcomes.

The configuration storage module 580 can store one or more sets of stimulation parameters that can be accessed, modified, or saved by the user or the system. These parameter sets may include predefined templates (e.g., standard clinical programs), user-defined configurations tailored to individual patient responses, or clinician-adjusted settings derived during programming sessions. Each stored configuration may define a combination of stimulation parameters, such as pulse width, amplitude, frequency, waveform shape, phase durations, electrode polarities, etc. In some examples, the configuration storage module 580 may also maintain associated metadata including timestamps, clinician notes, patient identifiers, etc. The usage of configuration storage module 580 can support efficient retrieval and reapplication of stimulation profiles, facilitating reproducibility, personalization, and longitudinal tracking of therapy.

The user can adjust and evaluate candidate stimulation parameters through visual feedback presented on the GUI 530. These candidate stimulation parameters remain inactive until they are programmed in the neurostimulator 510 for application. Once a set of stimulation parameters is selected and confirmed by the user, the programmer 520 may transmit the set of stimulation parameters to the neurostimulator 510 for application. In some examples, the programmer 520 may also interrogate the neurostimulator 510 to retrieve information such as currently active stimulation parameters, battery status, and diagnostic or physiological measurements (e.g., sensed signals for closed-loop control).

In practice, the systems shown herein, such as the neuromodulation system 500, can vary in complexity, with additional functionality, more complex components, and the like. For example, there can be additional functionality within the programmer 520. Additional components can be included to implement security, redundancy, and the like.

The neuromodulation system 500 and any of the other systems described herein can be implemented in conjunction with any of the hardware components described herein, such as the computing systems described below (e.g., processing units, memory, and the like). In any of the examples herein, the stimulation parameters, waveform, target position, and the like can be stored in one or more computer-readable storage media or computer-readable storage devices. The technologies described herein can be generic to the specifics of operating systems or hardware and can be applied in any variety of environments to take advantage of the described features.

### Example Overall Method for Programming Stimulation Parameters

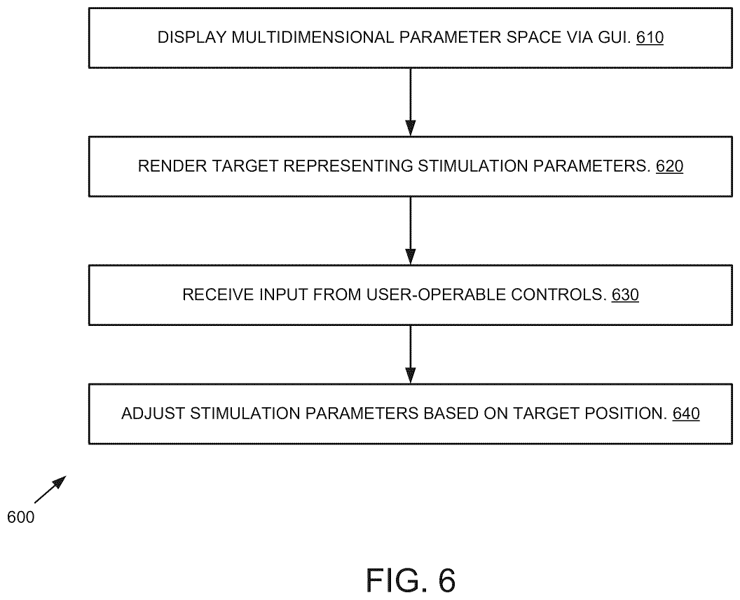

FIG. 6 is a flowchart illustrating an example overall method 600 for waveform editing and programming stimulation parameters of a neuromodulation system. The method 600 can be performed by the programmer 520 of FIG. 5.

At step 610, the method can display, via a graphical user interface (e.g., the GUI 530), a multidimensional parameter space. Each axis of the multidimensional parameter space corresponds to a respective stimulation parameter associated with stimulation pulses deliverable by the neuromodulation system.

At step 620, the method can render, within the multidimensional parameter space, a target representing a set of stimulation parameters corresponding to coordinates along two or more axes of the multidimensional parameter space.

At step 630, the method can receive, via one or more user-operable controls (e.g., the controls 540), input to move the target within the multidimensional parameter space.

At step 640, in response to movement of the target, the method can simultaneously adjust the stimulation parameters associated with the neuromodulation system along the two or more axes based on a position of the target in the multidimensional parameter space.

In some examples, axes of the multidimensional parameter space represent a combination of two or more stimulation parameters including: a total pulse width of a stimulation pulse, a proportion of the total pulse width attributed to a specific phase of the stimulation pulse, a stimulation amplitude, a stimulation frequency, a waveform shape, and an interphase characteristic defining a transition between distinct phases of the stimulation pulse.

In some examples, the multidimensional parameter space is a two-dimensional parameter space including two axes.

In some examples, the two axes include a first axis corresponding to a total pulse width of a stimulation pulse, and a second axis corresponding to a proportion of the total pulse width attributed to a specific phase of the stimulation pulse.

In some examples, the multidimensional parameter space is a three-dimensional parameter space comprising three axes.

In some examples, the two axes include a first axis corresponding to a total pulse width of a stimulation pulse, a second axis corresponding to a proportion of the total pulse width attributed to a specific phase of the stimulation pulse, and a third axis corresponding to an amplitude of the stimulation pulse.

In some examples, the method can further display, via the graphical user interface, one or more zones within the multidimensional parameter space. Each zone can correspond to a combination of stimulation parameters associated with a predefined stimulation effect.

In some examples, the method can further generate (e.g., using the simulator 560) the one or more zones based on outputs of one or more simulation models (e.g., models 570) configured to predict neural responses to combinations of stimulation parameters.

In some examples, the method can provide, via the graphical user interface, instant visual feedback representing the stimulation parameters along the two or more axes as the target is moved within the multidimensional parameter space.

In some examples, the target is one of a plurality of targets rendered within the multidimensional parameter space. Each target can represent a corresponding set of stimulation parameters saved by a user. The method can further receive user input to select one of the plurality of targets and edit the corresponding set of stimulation parameters by moving the selected target within the multidimensional parameter space.

In some examples, the method can receive user input via the one or more user-operable controls to change at least one stimulation parameter that is not represented by any of the axes of the multidimensional parameter space. In some examples, the method can further provide, via the graphical user interface, visual feedback indicating a current setting of the at least one stimulation parameter as it is changed.

The method 600, and any of the other methods described herein can be performed by computer-executable instructions (e.g., causing a computing system to perform the method) stored in one or more computer-readable media (e.g., storage or other tangible media) or stored in one or more computer-readable storage devices. Such methods can be performed in software, firmware, hardware, or combinations thereof. Such methods can be performed at least in part by a computing system (e.g., one or more computing devices).

The illustrated actions can be described from alternative perspectives while still implementing the technologies. For example, “receive” can also be described as “send” from a different perspective.

### Example Two-Dimensional Graphical User Interface for Waveform Editing

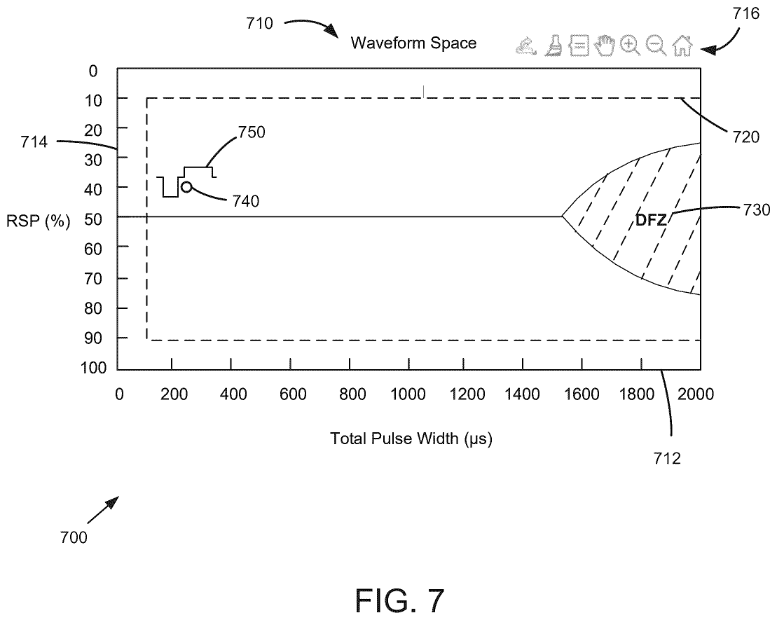

FIG. 7 illustrates an example GUI 700 configured for waveform editing in a two-dimensional stimulation parameter space 710 (also referred to as the “waveform space”). This interface allows a user to visualize and interact with stimulation parameters that define the shape and characteristics of electrical pulses to be delivered by a neuromodulation system. In FIG. 7, a horizontal axis 712 represents the total pulse width (in microseconds), and a vertical axis 714 represents the relative stimulation phase (RSP), which, in this example, corresponds to the percentage of the total pulse width attributed to the cathodic phase of the stimulation pulse. In other examples, the axes may be switched or configured to represent other stimulation parameters, such as amplitude, frequency, interphase delay, waveform shape characteristics, etc.

Within the stimulation parameter space 710, a boundary 720 defines the permissible region in which the user can adjust stimulation parameters. For example, FIG. 7 shows a boundary constraining total pulse width between approximately 100 and 2000 microseconds, and RSP between approximately 10% and 90%. These constraints may be predefined by clinical safety limits, device specifications, and/or user preferences. The user can manipulate a target 740, which represents a specific combination of stimulation parameters, within this bounded space. The target 740 can be represented as a cursor or a specific visual object. As the target 740 moves, both the total pulse width and the RSP are adjusted simultaneously.

A waveform preview 750 of the stimulation pulse corresponding to the current target position can be shown adjacent to the target 740. In some examples, the waveform preview 750 can represent one period of the stimulation pulse (or wavelet), updated dynamically to reflect changes in the parameter values determined by the position of the target 740. As a result, the user receives continuous visual feedback when exploring the stimulation parameter space 710, allowing for intuitive evaluation of waveform shape and timing characteristics of the stimulation pulses. In lieu of or in addition to the visual waveform preview 750, textual information may also be provided to display the current values of the relevant stimulation parameters.

In some examples, the GUI 700 may include one or more zones 730, such as a dual-firing zone (DFZ), visually marked within the stimulation parameter space 710. These zones may be generated through simulation based on physiological models and represent regions associated with particular stimulation effects, such as dual firing of axons, ECAP generation, and other therapeutic outcomes. The DFZ, for example, may correspond to stimulation parameter combinations that are predicted to induce a single neuron to fire twice in response to each delivered stimulation pulse, a phenomenon that may have therapeutic and safety implications depending on the context. Depending on the programming goals, the user may choose to move the target 740 into or out of a given zone. For example, entering a zone may be desirable to achieve a specific therapeutic effect, while in other cases, it may be beneficial to avoid certain regions to minimize side effects or undesired outcomes.

In some examples, the GUI 700 also includes additional user controls 716, which may support common interface operations such as zooming, panning, undo/redo, resetting the view to a home position, or the like. These controls can facilitate navigation and interaction within the stimulation parameter space 710. Although not explicitly shown in FIG. 7, other user-operable controls may also be available to enable direct manipulation of the target 740, including keyboard commands, touchscreen gestures, joystick input, and/or mouse-based dragging.

Thus, through the interactive GUI 700, the user can efficiently navigate the stimulation parameter space 710, with the ability to adjust multiple stimulation parameters (e.g., total pulse width and RSP) simultaneously by moving the target 740 within the defined boundary 720. As the target 740 is repositioned, the waveform preview 750 and model-informed feedback enable the user to visualize the impact of parameter changes instantly. This intuitive interface supports rapid exploration and informed decision-making for programming stimulation settings in a manner that accounts for therapeutic objectives, physiological constraints, and patient-specific considerations.

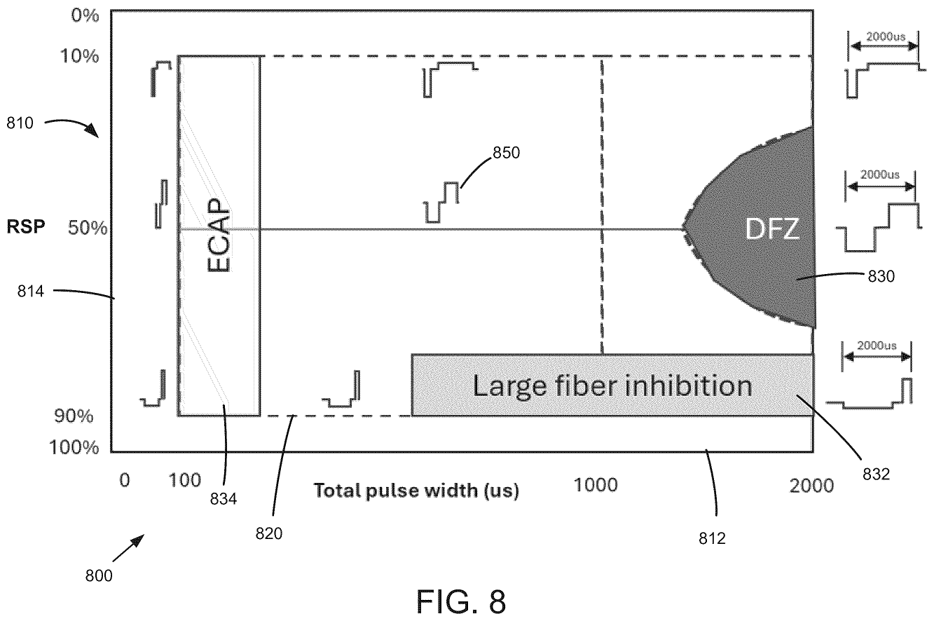

FIG. 8 illustrates another example GUI 800 configured for waveform editing and stimulation parameter programming. The GUI 800 presents a two-dimensional stimulation parameter space 810, which includes a boundary 820 that defines the permissible region within which the user can adjust stimulation parameters. While similar in format to the stimulation parameter space 710 shown in FIG. 7, the stimulation parameter space 810 further incorporates additional visual indicators and physiologically meaningful zones that provide enhanced guidance during waveform configuration. As with prior examples, the horizontal axis 812 represents the total pulse width (in microseconds), and the vertical axis 814 represents the RSP (ranging from 0% to 100%), corresponding to the proportion of the pulse duration attributed to the cathodic phase.

FIG. 8 shows a series of example waveform previews 850 at different coordinate locations across the stimulation parameter space 810. These waveform previews 850 illustrate example stimulation pulses that may be generated at different positions within the stimulation parameter space 810 as the user moves a target, rather than representing waveforms displayed simultaneously in the live GUI 800. For instance, the previews on the left correspond to shorter total pulse widths, while those in the center and on the right represent progressively longer pulse widths (e.g., 2000 μs). Vertically, the waveforms at the top of the space reflect lower RSP values (i.e., shorter cathodic phases relative to the total pulse width), while those in the middle and bottom correspond to increasing RSP values, including more dominant cathodic components.

FIG. 8 also illustrates several physiologically relevant zones within the stimulation parameter space 810. On the far right, a dual-firing zone (DFZ) 830 (similar to 730) is shown, representing a region where stimulation parameters are predicted to cause a neuron to fire twice in response to a single stimulation pulse. This effect may be desirable in certain therapeutic contexts or avoided depending on patient response. Toward the lower portion of the space, a horizontally extended large fiber inhibition zone 832 identifies parameter combinations that are predicted to selectively inhibit large-diameter fibers, which may be useful for minimizing side effects and/or enhancing selective targeting of smaller fibers. On the far left, an ECAP zone 834 highlights a vertical band or window where evoked compound action potentials (ECAPs) are likely to be detected. ECAPs may serve as objective indicators of neural recruitment and can assist in programming and verifying stimulation efficacy. As described above, these zones can be generated from model-based simulations.

Although a specific target or cursor is not depicted in FIG. 8, a user operating within GUI 800 may control a movable target similar to that shown in FIG. 7, navigating the stimulation parameter space 810 to explore and select stimulation parameters. As the target moves through the space, waveform previews (e.g., 850) may update in real time to reflect the current pulse width and RSP of the stimulation pulses, and the user can make informed adjustments based on proximity to visualized zones like ECAP zone 834, DFZ 830, and/or the large fiber inhibition zone 832.

### Example User-Operable Controls

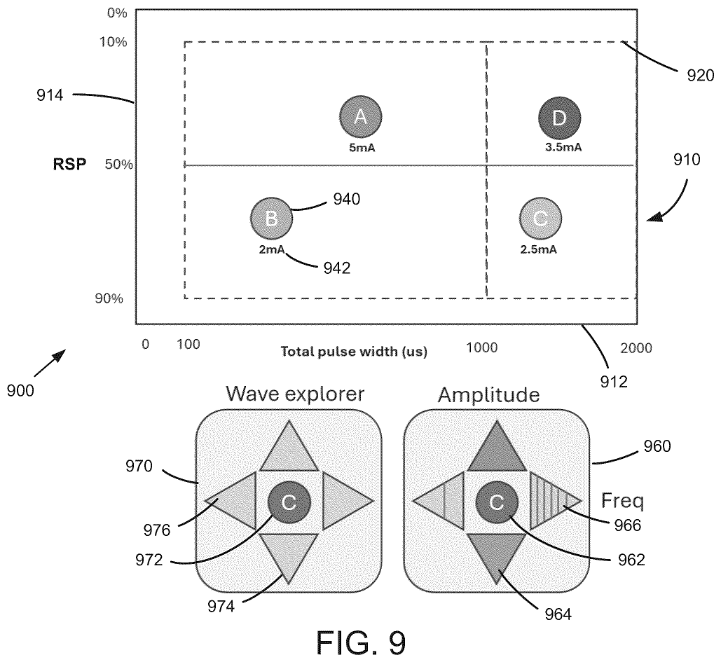

FIG. 9 illustrates an example GUI 900 configured to support advanced user-operable controls for editing stimulation parameters. The GUI 900 displays a two-dimensional stimulation parameter space 910 defined by a boundary 920, with horizontal axis 912 and vertical axis 914 representing total pulse width and RSP, respectively. For simplicity, no modeling zones are shown in this example, although it should be understood that one or more zones can be added to the stimulation parameter space 910, as described above.

Within the stimulation parameter space 910, a plurality of targets 940 (e.g., four such targets labeled A, B, C, and D) can be displayed simultaneously. Each target 940 represents a corresponding set of stimulation parameters, which may be predefined by the system or previously saved by a user (e.g., retrieved from configuration storage module 580). Each target 940 may also be associated with displayed information 942, such as a visual representation of the corresponding waveform or numerical data—in this example, the stimulation amplitude in milliamps (e.g., “2 mA”, “3.5 mA”, etc.). These targets 940 allow the user to compare, select, and refine different parameter configurations directly within the stimulation parameter space 910.

To interact with the targets, the system can provide one or more user-operable controls that may be implemented as physical interfaces (e.g., consoles, keyboards, dials, gesture controls) or integrated directly within the GUI (e.g., touchscreen widgets or virtual joysticks). Two such example controls are illustrated in FIG. 9: an amplitude-and-frequency control 960 for adjusting stimulation amplitude and/or frequency, and a wave explorer control 970 for editing waveform shape.

In FIG. 9, each control 960 and 970 includes a central button (e.g., button 962 for the amplitude-and-frequency control and button 972 for the wave explorer control) that visually identifies the currently selected target (e.g., displaying “C” to indicate that target C is active). In some implementations, in addition to serving as visual indicators, these central buttons can also be configured to select a target for editing. For example, pressing the central button 962 or 972 may cycle through all available targets 940, looping forward through the list. When a new target is selected, the display on both controls can be synchronized to reflect the same active target, ensuring consistency across different control interfaces.

Each control includes directional interface elements that enable distinct types of interactions. For example, in the amplitude-and-frequency control 960, a first pair of arrow buttons 966 can be used to adjust stimulation frequency up or down for the selected target. A second pair of buttons 964 can be configured to incrementally adjust (e.g., increase or decrease) stimulation amplitude. In some examples, the GUI 900 may render updated informational cues or visual feedback, such as numerical values or waveform previews, based on changes to the amplitude and/or frequency for the selected target, providing users with immediate confirmation of their input.

In the depicted example, the wave explorer control 970 includes two pairs of arrow buttons that enable manipulation of the currently selected target 940 within the two-dimensional stimulation parameter space 910. For instance, one pair of buttons 974 can be configured to move the selected target along the vertical axis 914 (thus adjusting RSP), while the other pair of buttons 976 can be configured to move the selected target along the horizontal axis 912 (thus adjusting the total pulse width). Thus, the user can reposition the selected target dynamically (e.g., in response to patient feedback on the perceived efficacy or comfort of the stimulation), resulting in the simultaneous adjustment of multiple stimulation parameters in a clinically responsive and intuitive manner.

As the user modifies the position of a target and/or adjusts its associated stimulation parameters using these controls, the updated set of parameters can be saved back to configuration storage (e.g., module 580), ensuring persistence across programming sessions. Additionally, the finalized parameters may be transmitted to the neurostimulator to effectuate real-time changes in delivered stimulation, thereby closing the loop between programming and therapy delivery.

Although FIG. 9 illustrates only two example controls (960 and 970) with specific button configurations, these implementations are merely illustrative and not limiting. Any number and variety of user-operable controls may be provided to support adjustment of other stimulation parameters not represented along the primary axes of the stimulation parameter space 910, such as interphase duration, electrode polarity, pulse waveform, etc. For example, a dedicated waveform control may be included to enable the user to select between different pulse shapes (e.g., square, sinusoidal, triangular, or sawtooth waveforms), either through discrete selections or continuous curve-based transformations. In some implementations, the central buttons (e.g., 962, 972) may serve solely as visual indicators of the currently selected target, without selection functionality. Instead, target selection can be performed directly through interaction with the GUI 900, for example, by tapping on a target displayed on a touchscreen. In such cases, certain physical controls, such as the wave explorer control 970, may be optional, as the user can reposition the selected target within the stimulation parameter space simply by dragging it on the screen. In some implementations, the central buttons (e.g., 962, 972) may also be optional, and the selected target may be visually indicated using alternative graphical techniques, such as highlighting the selected target, dimming or graying out unselected targets, outlining the active target, or applying other visual cues. In some examples, these user-operable controls may be implemented using a variety of physical or virtual modalities, including rotary dials, sliders, touchscreen widgets, gesture-recognition interfaces, or voice commands, depending on the system's hardware configuration and clinical workflow requirements.

### Example Three-Dimensional Graphical User Interface and User-Operable Controls

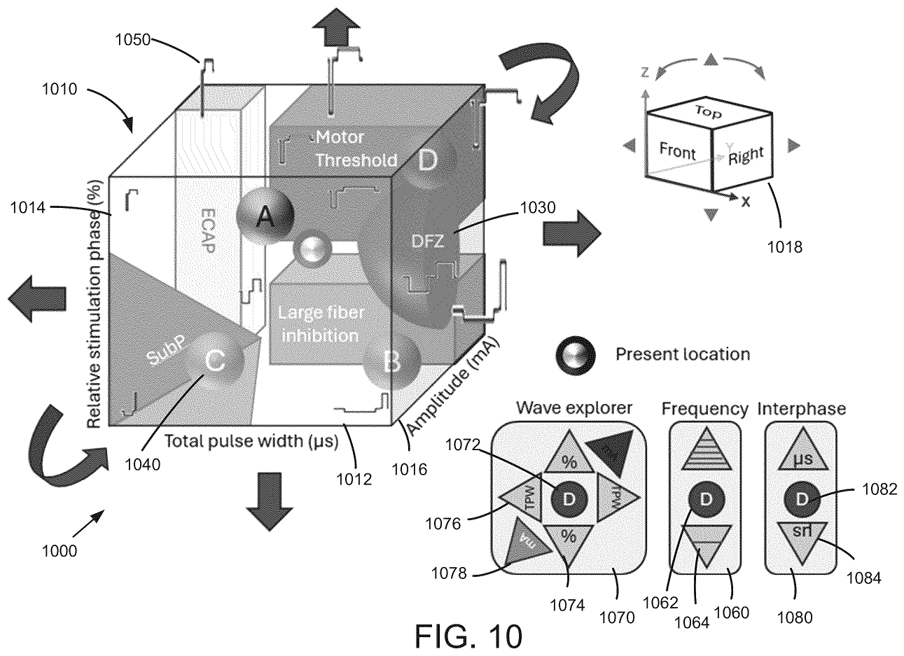

FIG. 10 illustrates another example GUI 1000 configured to display and navigate a three-dimensional (3D) stimulation parameter space 1010. Unlike earlier two-dimensional interfaces, this example introduces a third axis, providing users with a more comprehensive and spatially enriched view of the relationships between multiple stimulation parameters. In the depicted example, the axes of the parameter space include a horizontal axis 1012 representing total pulse width (in microseconds), a vertical axis 1014 representing RSP (expressed as a percentage), and a depth axis 1016 representing stimulation amplitude (e.g., in milliamps). This 3D space allows users to visualize and manipulate combinations of three stimulation parameters simultaneously.

Within the 3D stimulation parameter space 1010, the GUI 1000 can display one or more predictive physiological zones 1030. As described above, these zones may be generated via simulations based on physiological models and can be used to guide user decisions. In the depicted example, a dual-firing zone (DFZ) identifies parameter regions where neurons may fire twice per stimulation pulse. An ECAP zone indicates where evoked compound action potentials are likely to occur. A large fiber inhibition zone represents combinations of stimulation parameters that are expected to suppress large-diameter fiber activity. A motor threshold zone marks stimulation levels likely to activate motor fibers (e.g., helping avoid unintended motor responses). Additionally, a sub-paresthetic (SubP) zone represents parameter combinations predicted to produce therapeutic effects without eliciting perceptible sensations, thereby potentially enhancing patient comfort and overall tolerability.

Also shown within the stimulation parameter space 1010 are a plurality of targets 1040 (four are shown in FIG. 10, labeled A, B, C, and D). Each target represents a saved or user-defined combination of stimulation parameters. The user can select any of these targets for review or modification using the provided controls. A waveform preview 1050 may be shown alongside each target to visually represent the corresponding stimulation pulse, allowing real-time feedback as parameters are adjusted.

To support interaction with this multidimensional interface, optionally a visual orientation cube 1018 can be provided, which can serve as a view navigator. For instance, the orientation cube 1018 can help the user understand the current orientation of the 3D space and can optionally be interactive, enabling the user to rotate or reorient the stimulation parameter space 1010 for more intuitive exploration of complex zones and parameter relationships.

FIG. 10 also shows a set of user-operable controls for adjusting stimulation parameters. In the depicted example, a wave explorer control 1070 is configured to enable spatial navigation within the 3D stimulation parameter space 1010. It includes a central button 1072 which can be used to identify or switch the currently selected target (e.g., target D in the depicted example). For instance, pressing the central button 1072 may cycle through available targets, and the selection can be synchronized across all other controls to ensure consistent display and editing of the same target. Surrounding the central button 1072 are three pairs of directional arrow buttons: buttons 1074 (e.g., for navigating total pulse width along the horizontal axis 1012), buttons 1076 (e.g., for adjusting RSP along the vertical axis 1014), and buttons 1078 (for modifying amplitude along the depth axis 1016). These buttons allow the user to move the selected target 1040 within the stimulation parameter space 1010, thereby simultaneously updating all three associated stimulation parameters in real time.

A frequency control 1060 provides buttons for adjusting the stimulation frequency of the selected target. The central button 1062 identifies the active target and, when pressed, may also be used to cycle through the available targets, with the selection kept in sync with the other controls. An adjacent pair of arrow buttons 1064 allows the user to increase or decrease the stimulation frequency. Similarly, an interphase control 1080 can be configured for interphase timing adjustments. It includes a central button 1082 for identifying (and optionally switching) the active target, and a pair of arrow buttons 1084 that allow the user to modify the interphase duration, such as the time interval between the cathodic and anodic phases of the stimulation pulse. Together, the frequency control 1060 and interphase control 1080 facilitate refinement of parameters beyond those directly represented along the 3D spatial axes.

While FIG. 10 illustrates specific button-based controls for navigating and adjusting stimulation parameters, these are merely examples and not limiting. In alternative implementations, some or all of the functions provided by controls 1060, 1070, and 1080 may be integrated into a touchscreen GUI, for instance, allowing the user to tap or drag a target directly within the 3D stimulation parameter space 1010. Target selection could also be performed via gesture input, voice command, or external input devices such as joysticks or rotary dials. Visual cues such as highlighting, outlining, or dimming may be used to indicate the active target in lieu of or in addition to central button indicators.

### Example Navigation of N-dimensional Parameter Space

In some embodiments, the GUI can be configured to enable practical navigation and visualization of an n-dimensional stimulation parameter space, where n>3. Although the primary GUI may display only three axes at a time (e.g., total pulse width, relative stimulation phase, and amplitude), users can configure which parameters are mapped to each axis via selectable interface elements (e.g., dropdown menus located near each axis, or the like). This allows users to explore different combinations of parameters by dynamically reassigning dimensions without changing the core structure of the GUI.

When a user selects a new parameter for a given axis, any visual zones of interest (e.g., dual-firing zones, ECAP zones, etc.) can be automatically repositioned within the 3D space to reflect their correct location based on the newly defined axes. In some implementations, the GUI may render multiple interactive 3D parameter spaces (“cubes”) simultaneously, each with its own axis definitions. Users can switch between these cubes to manipulate different subsets of parameters, with all views kept synchronized in real time. As such, this modular, axis-redefinable framework supports efficient exploration and editing of high-dimensional stimulation settings in an intuitive and spatially organized manner.

### Example Virtual GUI Access and AI-Controlled Programming

In some embodiments, the disclosed technologies can support programming of stimulation parameters via a virtual GUI-a non-visual, machine-readable representation of the multidimensional parameter space. This virtual GUI, which may correspond to the GUI 530 described above (see FIG. 5), can be accessed programmatically by an artificial intelligence (AI) agent without requiring physical or visual instantiation. Rather than interacting with a rendered display, the AI agent can be configured to read from and write to data structures that define the parameter axes, current target positions, stimulation zones, boundaries, and configuration constraints. This allows navigation, evaluation, and manipulation of the parameter space using computational means alone.

The AI agent may be implemented as software running locally on the programmer 520 or remotely via a networked platform, and may incorporate rule-based systems, numerical optimizers, and/or machine learning models (e.g., reinforcement learning, supervised models trained on patient response data, etc.). Operating within the virtual GUI, the AI agent can analyze mathematical descriptions of the stimulation space and autonomously update stimulation settings based on clinical inputs such as physiological feedback, verbal reports from the patient, and/or sensed signals. By simulating the effects of candidate configurations and referencing modeled zones of interest, the AI agent can iteratively optimize parameter combinations to enhance therapeutic outcomes and/or reduce side effects, thereby functioning as a closed-loop digital therapy planner without requiring conventional GUI interaction.

### Example Advantages

The disclosed waveform editing technologies for neuromodulation systems offer significant technical and clinical advantages over conventional approaches, particularly in how stimulation parameters are visualized, manipulated, and optimized.

Conventional systems typically restrict the user to modifying one stimulation parameter at a time, such as amplitude, pulse width, or frequency, which can make comprehensive waveform exploration slow and inefficient. In contrast, the disclosed technologies provide a multidimensional parameter space, where users can simultaneously adjust multiple stimulation parameters by repositioning a target within the space. This spatial interface enables intuitive and accelerated exploration of the stimulation parameter landscape, dramatically reducing the time and complexity required to identify effective therapeutic settings.

Unlike traditional programming interfaces, which offer limited or tabular access to discrete parameter values, the disclosed system presents a continuous, visual representation of the entire available waveform space. This allows users to see not only where they are within the parameter landscape, but also how different parameter combinations relate to each other in terms of physiological/therapeutic effects and safety.

Moreover, the disclosed technologies support integration of computational physiological models to generate model-based zones of interest within the stimulation parameter space. These zones may correspond to regions where specific neural behaviors or therapeutic effects are predicted to occur. By visually representing these zones within the GUI, the system offers real-time, simulation-driven guidance that enables users to steer stimulation parameters toward or away from regions associated with desired clinical outcomes or potential side effects. This model-informed interface transforms the user's programming experience from manual trial-and-error into a guided and efficient therapeutic optimization process.

Additionally, the disclosed technologies allow preconfigured or saved targets to be loaded and compared visually, supporting rapid selection or refinement of previously saved stimulation settings. Targets can also be updated dynamically based on real-time patient feedback and/or physiological measurements.

Further, the GUIs disclosed herein have broad accessibility and adaptability across user types and clinical contexts. Clinicians can leverage intuitive visuals to simplify complex stimulation parameters during programming. Patients may interact with simplified or mobile versions to adjust therapy within safe, predefined limits. By abstracting technical details into user-friendly graphical elements, the system empowers even non-specialists to navigate and personalize stimulation settings effectively.

Last but not least, the disclosed waveform editing tool can serve as a control layer atop any stimulation platform through application programming interfaces (APIs) or other communication interfaces, effectively decoupling the interface from underlying hardware. This modularity allows the same GUI framework to be deployed across different implantable pulse generators, neurostimulators, or external stimulators, bringing its advantages to a broad array of neuromodulation systems.

### Example Computing Systems

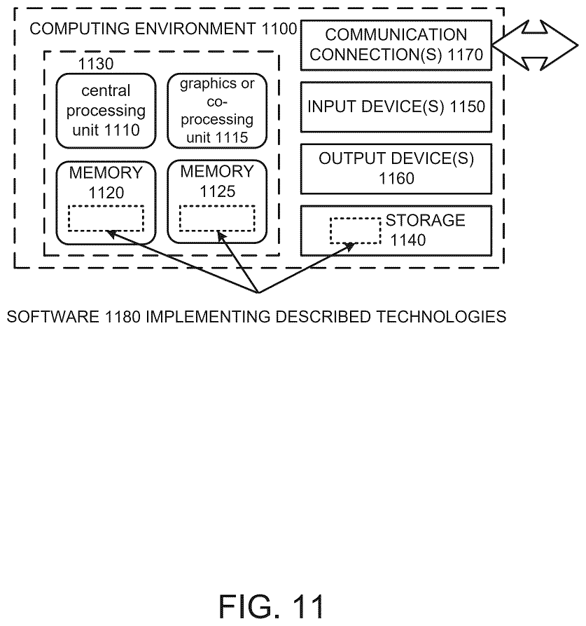

The innovations described herein can be implemented by the computing system 1100 described depicted in FIG. 11. The computing system 1100 is not intended to suggest any limitation as to scope of use or functionality of the present disclosure, as the innovations can be implemented in diverse computing systems.

With reference to FIG. 11, the computing system 1100 includes one or more processing units 1110, 1115 and memory 1120, 1125. In FIG. 11, this basic configuration 1130 is included within a dashed line. The processing units 1110, 1115 can execute computer-executable instructions, such as for implementing the features described in the examples herein. A processing unit can be a general-purpose central processing unit (CPU), processor in an application-specific integrated circuit (ASIC), or any other type of processor. In a multi-processing system, multiple processing units can execute computer-executable instructions to increase processing power. For example, FIG. 11 shows a central processing unit 1110 as well as a graphics processing unit or co-processing unit 1115. The tangible memory 1120, 1125 can be volatile memory (e.g., registers, cache, RAM), non-volatile memory (e.g., ROM, EEPROM, flash memory, etc.), or some combination of the two, accessible by the processing unit(s) 1110, 1115. The memory 1120, 1125 can store software 1180 implementing one or more innovations described herein, in the form of computer-executable instructions suitable for execution by the processing unit(s) 1110, 1115.

The computing system 1100 can have additional features. For example, the computing system 1100 can include storage 1140, one or more input devices 1150, one or more output devices 1160, and one or more communication connections 1170, including input devices, output devices, and communication connections for interacting with a user. An interconnection mechanism (not shown) such as a bus, controller, or network can interconnect the components of the computing system 1100. Typically, operating system software (not shown) can provide an operating environment for other software executing in the computing system 1100, and coordinate activities of the components of the computing system 1100.

The tangible storage 1140 can be removable or non-removable, and can include magnetic disks, magnetic tapes or cassettes, CD-ROMs, DVDs, or any other medium which can be used to store information in a non-transitory way and which can be accessed within the computing system 1100. The storage 1140 can store instructions for the software or method (e.g., the beamforming) implementing one or more innovations described herein.

The input device(s) 1150 can be an input device such as a keyboard, mouse, pen, or trackball, a voice input device, a scanning device, touch device (e.g., touchpad, display, or the like) or another device that provides input to the computing system 1100. The output device(s) 1160 can be a display, printer, speaker, CD-writer, or another device that provides output from the computing system 1100.

The communication connection(s) 1170 can enable communication over a communication medium to another computing entity. The communication medium can convey information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

The innovations can be described in the context of computer-executable instructions, such as those included in program modules, being executed in a computing system on a target real or virtual processor (e.g., which is ultimately executed on one or more hardware processors). Generally, program modules or components include routines, programs, libraries, objects, classes, components, data structures, etc. that perform particular tasks or implement particular abstract data types. The functionality of the program modules can be combined or split between program modules as desired in various embodiments. Computer-executable instructions for program modules can be executed within a local or distributed computing system.

For the sake of presentation, the detailed description uses terms like “determine” and “use” to describe computer operations in a computing system. These terms are high-level descriptions for operations performed by a computer, and should not be confused with acts performed by a human being. The actual computer operations corresponding to these terms vary depending on implementation.

### Computer-Readable Media

Any of the computer-readable media herein can be non-transitory (e.g., volatile memory such as DRAM or SRAM, nonvolatile memory such as magnetic storage, optical storage, or the like) and/or tangible. Any of the storing actions described herein can be implemented by storing in one or more computer-readable media (e.g., computer-readable storage media or other tangible media). Any of the things (e.g., data created and used during implementation) described as stored can be stored in one or more computer-readable media (e.g., computer-readable storage media or other tangible media). Computer-readable media can be limited to implementations not consisting of a signal.

Any of the methods described herein can be implemented by computer-executable instructions in (e.g., stored on, encoded on, or the like) one or more computer-readable media (e.g., computer-readable storage media or other tangible media) or one or more computer-readable storage devices (e.g., memory, magnetic storage, optical storage, or the like). Such instructions can cause a computing device to perform the method. The technologies described herein can be implemented in a variety of programming languages.

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art. Any theories of operation are to facilitate explanation, but the disclosed systems, methods, and apparatus are not limited to such theories of operation.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (e.g., mechanically or chemically) coupled or linked and does not exclude the presence of intermediate elements between the coupled or associated items absent specific contrary language.

Directions and other relative references (e.g., inner, outer, upper, lower, etc.) may be used to facilitate discussion of the drawings and principles herein, but are not intended to be limiting. For example, certain terms may be used such as “inside,” “outside,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

In any of the examples described herein, an operation performed in real-time means that the operation can be completed with negligible processing latency (e.g., the operation can be completed within 1 second, etc.).

### Example Clauses

Any of the following clauses can be implemented.

Clause 1. A computing system for programming stimulation parameters of a neuromodulation system, the computing system comprising: a graphical user interface configured to: display a multidimensional parameter space, wherein each axis of the multidimensional parameter space corresponds to a respective stimulation parameter associated with stimulation pulses deliverable by the neuromodulation system; and render a target within the multidimensional parameter space, the target representing a set of stimulation parameters corresponding to coordinates along two or more axes of the multidimensional parameter space; and one or more user-operable controls configured to enable movement of the target within the multidimensional parameter space; wherein movement of the target within the multidimensional parameter space causes simultaneous adjustment of the stimulation parameters associated with the neuromodulation system along the two or more axes based on a position of the target in the multidimensional parameter space.

Clause 2. The computing system of clause 1, wherein the multidimensional parameter space is a two-dimensional parameter space comprising two axes.

Clause 3. The computing system of clause 2, wherein the two axes comprise a first axis corresponding to a total pulse width of a stimulation pulse, and a second axis corresponding to a proportion of the total pulse width attributed to a specific phase of the stimulation pulse.

Clause 4. The computing system of clause 1, wherein the multidimensional parameter space is a three-dimensional parameter space comprising three axes.

Clause 5. The computing system of any one of clauses 1-4, wherein the graphical user interface is further configured to display one or more zones within the multidimensional parameter space, each zone corresponding to a combination of stimulation parameters associated with a predefined stimulation effect.

Clause 6. The computing system of clause 5, wherein the one or more zones are generated based on outputs of one or more simulation models configured to predict neural responses to combinations of stimulation parameters.

Clause 7. The computing system of any one of clauses 1-6, wherein the graphical user interface is further configured to provide instant visual feedback representing the stimulation parameters along the two or more axes as the target is moved within the multidimensional parameter space.

Clause 8. The computing system of any one of clauses 1-7, wherein the target is one of a plurality of targets rendered within the multidimensional parameter space, each target representing a corresponding set of stimulation parameters saved by a user; and wherein the one or more user-operable controls are configured to enable the user to select any one of the plurality of targets and to edit the corresponding set of stimulation parameters by moving the selected target within the multidimensional parameter space.

Clause 9. The computing system of any one of clauses 1-8, wherein the one or more user-operable controls are further configured to change at least one stimulation parameter that is not represented by any of the axes of the multidimensional parameter space; and wherein the graphical user interface is further configured to provide visual feedback indicating a current setting of the at least one stimulation parameter as it is changed.

Clause 10. The computing system of any one of clauses 1-9, wherein the axes of the multidimensional parameter space represent a combination of two or more stimulation parameters including: a total pulse width of a stimulation pulse, a proportion of the total pulse width attributed to a specific phase of the stimulation pulse, a stimulation amplitude, a stimulation frequency, a waveform shape, and an interphase characteristic defining a transition between distinct phases of the stimulation pulse.

Clause 11. A computer-implemented method for programming stimulation parameters of a neuromodulation system, the method comprising: displaying, via a graphical user interface, a multidimensional parameter space, wherein each axis of the multidimensional parameter space corresponds to a respective stimulation parameter associated with stimulation pulses deliverable by the neuromodulation system; rendering, within the multidimensional parameter space, a target representing a set of stimulation parameters corresponding to coordinates along two or more axes of the multidimensional parameter space; receiving, via one or more user-operable controls, input to move the target within the multidimensional parameter space; and in response to movement of the target, simultaneously adjusting the stimulation parameters associated with the neuromodulation system along the two or more axes based on a position of the target in the multidimensional parameter space.

Clause 12. The computer-implemented method of clause 11, wherein the multidimensional parameter space is a two-dimensional parameter space comprising two axes.

Clause 13. The computer-implemented method of clause 12, wherein the two axes comprise a first axis corresponding to a total pulse width of a stimulation pulse, and a second axis corresponding to a proportion of the total pulse width attributed to a specific phase of the stimulation pulse.

Clause 14. The computer-implemented method of clause 11, wherein the multidimensional parameter space is a three-dimensional parameter space comprising three axes.

Clause 15. The computer-implemented method of any one of clauses 11-14, further comprising displaying, via the graphical user interface, one or more zones within the multidimensional parameter space, each zone corresponding to a combination of stimulation parameters associated with a predefined stimulation effect.

Clause 16. The computer-implemented method of clause 15, further comprising generating the one or more zones based on outputs of one or more simulation models configured to predict neural responses to combinations of stimulation parameters.

Clause 17. The computer-implemented method of any one of clauses 11-16, further comprising providing, via the graphical user interface, instant visual feedback representing the stimulation parameters along the two or more axes as the target is moved within the multidimensional parameter space.

Clause 18. The computer-implemented method of any one of clauses 11-17, wherein the target is one of a plurality of targets rendered within the multidimensional parameter space, each target representing a corresponding set of stimulation parameters saved by a user; the method further comprising receiving user input to select one of the plurality of targets and editing the corresponding set of stimulation parameters by moving the selected target within the multidimensional parameter space.

Clause 19. The computer-implemented method of any one of clauses 11-18, further comprising receiving user input via the one or more user-operable controls to change at least one stimulation parameter that is not represented by any of the axes of the multidimensional parameter space; and providing, via the graphical user interface, visual feedback indicating a current setting of the at least one stimulation parameter as it is changed.

Clause 20. One or more non-transitory computer-readable media having encoded thereon computer-executable instructions causing one or more processors to perform a method for programming stimulation parameters of a neuromodulation system, the method comprising: displaying, via a graphical user interface, a multidimensional parameter space, wherein each axis of the multidimensional parameter space corresponds to a respective stimulation parameter associated with stimulation pulses deliverable by the neuromodulation system; rendering, within the multidimensional parameter space, a target representing a set of stimulation parameters corresponding to coordinates along two or more axes of the multidimensional parameter space; receiving, via one or more user-operable controls, input to move the target within the multidimensional parameter space; and in response to movement of the target, simultaneously adjusting the stimulation parameters associated with the neuromodulation system along the two or more axes based on a position of the target in the multidimensional parameter space.

The technologies from any clause can be combined with the technologies described in any one or more of the other clauses.

In view of the many possible embodiments to which the principles of the disclosed technology can be applied, it should be recognized that the illustrated embodiments are examples of the disclosed technology and should not be taken as a limitation on the scope of the disclosed technology. Rather, the scope of the disclosed technology includes what is covered by the scope and spirit of the following claims.

What is claimed is:

1. A computing system for programming stimulation parameters of a neuromodulation system, the computing system comprising:
a graphical user interface configured to:
display a multidimensional parameter space, wherein each axis of the multidimensional parameter space corresponds to a respective stimulation parameter associated with stimulation pulses deliverable by the neuromodulation system; and
render a target within the multidimensional parameter space, the target representing a set of stimulation parameters corresponding to coordinates along two or more axes of the multidimensional parameter space; and
one or more user-operable controls configured to enable movement of the target within the multidimensional parameter space;
wherein movement of the target within the multidimensional parameter space causes simultaneous adjustment of the stimulation parameters associated with the neuromodulation system along the two or more axes based on a position of the target in the multidimensional parameter space.

2. The computing system of claim 1, wherein the multidimensional parameter space is a two-dimensional parameter space comprising two axes.

3. The computing system of claim 2, wherein the two axes comprise a first axis corresponding to a total pulse width of a stimulation pulse, and a second axis corresponding to a proportion of the total pulse width attributed to a specific phase of the stimulation pulse.

4. The computing system of claim 1, wherein the multidimensional parameter space is a three-dimensional parameter space comprising three axes.

5. The computing system of claim 1, wherein the graphical user interface is further configured to display one or more zones within the multidimensional parameter space, each zone corresponding to a combination of stimulation parameters associated with a predefined stimulation effect.

6. The computing system of claim 5, wherein the one or more zones are generated based on outputs of one or more simulation models configured to predict neural responses to combinations of stimulation parameters.

7. The computing system of claim 1, wherein the graphical user interface is further configured to provide instant visual feedback representing the stimulation parameters along the two or more axes as the target is moved within the multidimensional parameter space.

8. The computing system of claim 1, wherein the target is one of a plurality of targets rendered within the multidimensional parameter space, each target representing a corresponding set of stimulation parameters saved by a user; and wherein the one or more user-operable controls are configured to enable the user to select any one of the plurality of targets and to edit the corresponding set of stimulation parameters by moving the selected target within the multidimensional parameter space.

9. The computing system of claim 1, wherein the one or more user-operable controls are further configured to change at least one stimulation parameter that is not represented by any of the axes of the multidimensional parameter space; and wherein the graphical user interface is further configured to provide visual feedback indicating a current setting of the at least one stimulation parameter as it is changed.

10. The computing system of claim 1, wherein the axes of the multidimensional parameter space represent a combination of two or more stimulation parameters including: a total pulse width of a stimulation pulse, a proportion of the total pulse width attributed to a specific phase of the stimulation pulse, a stimulation amplitude, a stimulation frequency, a waveform shape, and an interphase characteristic defining a transition between distinct phases of the stimulation pulse.

11. A computer-implemented method for programming stimulation parameters of a neuromodulation system, the method comprising:
displaying, via a graphical user interface, a multidimensional parameter space, wherein each axis of the multidimensional parameter space corresponds to a respective stimulation parameter associated with stimulation pulses deliverable by the neuromodulation system;
rendering, within the multidimensional parameter space, a target representing a set of stimulation parameters corresponding to coordinates along two or more axes of the multidimensional parameter space;
receiving, via one or more user-operable controls, input to move the target within the multidimensional parameter space; and
in response to movement of the target, simultaneously adjusting the stimulation parameters associated with the neuromodulation system along the two or more axes based on a position of the target in the multidimensional parameter space.

12. The computer-implemented method of claim 11, wherein the multidimensional parameter space is a two-dimensional parameter space comprising two axes.

13. The computer-implemented method of claim 12, wherein the two axes comprise a first axis corresponding to a total pulse width of a stimulation pulse, and a second axis corresponding to a proportion of the total pulse width attributed to a specific phase of the stimulation pulse.

14. The computer-implemented method of claim 11, wherein the multidimensional parameter space is a three-dimensional parameter space comprising three axes.

15. The computer-implemented method of claim 11, further comprising displaying, via the graphical user interface, one or more zones within the multidimensional parameter space, each zone corresponding to a combination of stimulation parameters associated with a predefined stimulation effect.

16. The computer-implemented method of claim 15, further comprising generating the one or more zones based on outputs of one or more simulation models configured to predict neural responses to combinations of stimulation parameters.

17. The computer-implemented method of claim 11, further comprising providing, via the graphical user interface, instant visual feedback representing the stimulation parameters along the two or more axes as the target is moved within the multidimensional parameter space.

18. The computer-implemented method of claim 11, wherein the target is one of a plurality of targets rendered within the multidimensional parameter space, each target representing a corresponding set of stimulation parameters saved by a user; the method further comprising receiving user input to select one of the plurality of targets and editing the corresponding set of stimulation parameters by moving the selected target within the multidimensional parameter space.

19. The computer-implemented method of claim 11, further comprising receiving user input via the one or more user-operable controls to change at least one stimulation parameter that is not represented by any of the axes of the multidimensional parameter space; and providing, via the graphical user interface, visual feedback indicating a current setting of the at least one stimulation parameter as it is changed.

20. One or more non-transitory computer-readable media having encoded thereon computer-executable instructions causing one or more processors to perform a method for programming stimulation parameters of a neuromodulation system, the method comprising:
displaying, via a graphical user interface, a multidimensional parameter space, wherein each axis of the multidimensional parameter space corresponds to a respective stimulation parameter associated with stimulation pulses deliverable by the neuromodulation system;
rendering, within the multidimensional parameter space, a target representing a set of stimulation parameters corresponding to coordinates along two or more axes of the multidimensional parameter space;
receiving, via one or more user-operable controls, input to move the target within the multidimensional parameter space; and
in response to movement of the target, simultaneously adjusting the stimulation parameters associated with the neuromodulation system along the two or more axes based on a position of the target in the multidimensional parameter space.
